(19) United States
(12) Patent Application Publication
(10) Pub. No.: US 2022/0218237 A1
(43) Pub. Date: Jul. 14, 2022

(54) NEEDLE-TYPE BIOSENSOR

(71) Applicant: LG ELECTRONICS INC., Seoul (KR)

(72) Inventors: Jungsun HAN, Seoul (KR); Seonghyok KIM, Seoul (KR); Jeankun OH, Seoul (KR)

(73) Assignee: LG ELECTRONICS INC., Seoul (KR)

(21) Appl. No.: 17/605,854

(22) PCT Filed: Jun. 19, 2019
(86) PCT No.: PCT/KR2019/007403

§ 371 Date: Oct. 22, 2021

(51) Int. Cl. A61B 5/1459; A61B 5/145; A61B 5/00; A61B 5/157

(52) U.S. Cl. CPC A61B 5/1459; A61B 5/14532; A61B 2562/12; A61B 5/157; A61B 5/685

(57) ABSTRACT

The present invention relates to an optical sensing-type needle-type biosensor, and provides a needle-type biosensor comprising: a hollow microneedle comprising an internal channel connected to a tip end portion; a fluorescent sensor provided in the internal channel and producing fluorescence by binding to a specific target material; and a reader for detecting the fluorescence from the fluorescent sensor, wherein the internal channel comprises a region where the diameter of one end thereof connected to the tip end portion is increased.

### TECHNICAL FIELD

The present disclosure relates to a needle-type biosensor. Specifically, the present disclosure may be applied to a technical field of fixing a fluorescence sensor to a hollow microneedle.

### BACKGROUND ART

Diabetic patients with impaired blood glucose control measure blood glucose thereof several times a day using a blood glucose meter and take medicine or inject themselves with insulin. Existing blood glucose meters using blood cause pain during measurement and at the same are not able to accurately determine a range of change in the blood glucose.

In addition, in a case of patients with a large blood glucose change range, drug delivery and preparation for the measurement results are difficult, so that a demand for development of a continuous glucose monitoring system (CGMS) is increasing.

Existing continuous glucose monitoring systems include a continuous glucose monitoring system of an electrochemical scheme and a continuous glucose monitoring system of an optical scheme.

The electrochemical scheme is a scheme of informing a result value by measuring an electrochemical signal after inserting an electrode to which an enzyme for the blood glucose measurement is applied into a body. The electrochemical scheme, which is the enzyme-based measurement scheme, has a disadvantage of not being able to be used for a long time (usable for up to 2 weeks at a current technology level).

The optical scheme is a scheme of informing the result value by sensing light emitted from an optical sensor. The optical scheme is advantageous over the electrochemical scheme in terms of long-term use and sensitivity. However, the existing optical scheme requires an incision procedure to insert or remove the optical sensor into or from the body, and has a limitation in a period of use of not being able to exceeding 180 days resulted from a limitation of a battery capacity. In relation, FIG. 1 shows an embodiment of the optical sensor that is inserted into the body in the continuous glucose monitoring system of the existing optical scheme, and FIG. 2 shows an embodiment of sensing the light emission of the optical sensor inserted into the body through an external reader.

Therefore, in recent years, a continuous glucose monitoring system of the optical scheme using a microneedle is being considered to overcome the limitation of the period of use and the disadvantage of requiring the incision procedure of the continuous glucose monitoring system of the existing optical scheme.

The continuous glucose monitoring system of the optical scheme using the microneedle has an advantage of reducing fear of the patients and a risk of injury and infection because of being able to be attached and detached without the incision procedure, and has an advantage of being able to be applied for a long time because battery replacement is easy.

However, there is a difficulty in fixing a blood glucose sensor to the microneedle in the continuous glucose monitoring system of the optical scheme using the microneedle, so that there is a need for a solution for this.

### DISCLOSURE

### Technical Problem

The present disclosure aims to address the aforementioned problems and other problems through the specification of the present disclosure as well.

The present disclosure aims to fix a fluorescence sensor substance to a hollow microneedle.

The present disclosure aims to quantitatively fix a fluorescence sensor substance to a hollow microneedle.

### Technical Solutions

According to one embodiment of the present disclosure to achieve the above purpose, provided is a needle-type biosensor including a microneedle containing a fluorescence receptor that fluoresces by selectively being coupled to a specific target substance at one end thereof inserted into skin, and a reader disposed at the other end of the microneedle to sense the specific target substance through light emitted by the fluorescent receptor.

In addition, according to one embodiment of the present disclosure, the fluorescence sensor may be gelled along a shape of the internal flow channel after being injected into the internal flow channel from said one end of the internal flow channel in a solution state, and the fluorescence sensor may be fixed to the internal flow channel by a difference in diameter of the internal flow channel.

In addition, according to one embodiment of the present disclosure, the internal flow channel may include a step where an internal diameter thereof varies discontinuously.

In addition, according to one embodiment of the present disclosure, the internal flow channel may include the step at a location a preset distance away from said one end of the internal flow channel, and the fluorescence sensor may be disposed from said one end of the internal flow channel to the step.

In addition, according to one embodiment of the present disclosure, the internal flow channel may have the section with the increasing diameter between said one end of the internal flow channel and the step.

In addition, according to another embodiment of the present disclosure, provided is a method for manufacturing a needle-type biosensor including manufacturing a hollow microneedle including an internal flow channel defined therein connected to a tip thereof, injecting a fluorescence sensor in a solution state into one end of the internal flow channel, and gelling and fixing the fluorescence sensor injected into the internal flow channel.

In addition, according to another embodiment of the present disclosure, the manufacturing of the hollow microneedle may include performing a hydrophilic treatment on the internal flow channel.

In addition, according to another embodiment of the present disclosure, the injecting of the fluorescence sensor may include injecting the fluorescence sensor in the solution state into the internal flow channel through a capillary phenomenon.

In addition, according to another embodiment of the present disclosure, the manufacturing of the hollow microneedle may include forming a section with a diameter increasing to be greater than a diameter of said one end of the internal flow channel connected to the tip.

In addition, according to another embodiment of the present disclosure, the gelling and fixing of the fluorescence sensor may include gelling the fluorescence sensor in the solution state using at least one of a temperature, a time, and a UV, and the internal flow channel may prevent the gelled fluorescence sensor from leaking with a difference in diameter of the internal flow channel.

In addition, according to another embodiment of the present disclosure, the manufacturing of the hollow microneedle may include forming a step where an internal diameter of the internal flow channel varies discontinuously, and the injecting of the fluorescence sensor may include injecting the fluorescence sensor from said one end of the internal flow channel to the step.

### Advantageous Effects

Effects of the needle-type biosensor according to the present disclosure will be described as follows.

In the present disclosure, the fluorescence sensor may be fixed to the hollow microneedle, so that the fluorescence sensor may be prevented from being introduced into the body while the hollow microneedle is inserted into the skin.

In the present disclosure, the concentration of the substance to be monitored may be measured through the degree of fluorescing by quantitatively fixing the fluorescence sensor to the hollow microneedle.

Further scope of applicability of the present disclosure will become apparent from a detailed description below. However, various changes and modifications within the spirit and scope of the present disclosure may be clearly understood by those skilled in the art, so that it should be understood that the detailed description and the specific embodiments, such as preferred embodiments of the present disclosure, are given by way of example only

### DESCRIPTION OF DRAWINGS

FIG. 1 shows an embodiment of an optical sensor that is inserted into a body in a continuous glucose monitoring system of an existing optical scheme.

FIG. 2 shows an embodiment of sensing light emission of an optical sensor inserted into a body through an external reader.

FIG. 3 shows an embodiment of a needle-type biosensor according to the present disclosure.

FIG. 4 is an enlarged view of a reader in FIG. 3.

FIGS. 5 and 6 show other embodiments of a needle-type biosensor according to the present disclosure.

FIGS. 7 and 8 are views for illustrating a method for introducing a fluorescence sensor into a hollow microneedle in a needle-type biosensor according to the present disclosure.

FIG. 9 is a view for illustrating a structure of a hollow microneedle for fixing an introduced fluorescence sensor in a needle-type biosensor according to the present disclosure.

FIG. 10 is a view for illustrating a structure of a hollow microneedle for quantitatively introducing a fluorescence sensor in a needle-type biosensor according to the present disclosure.

FIG. 11 is a view for illustrating data sensed through a reader based on a degree of light emission of a fluorescent substance in a needle-type biosensor according to the present disclosure.

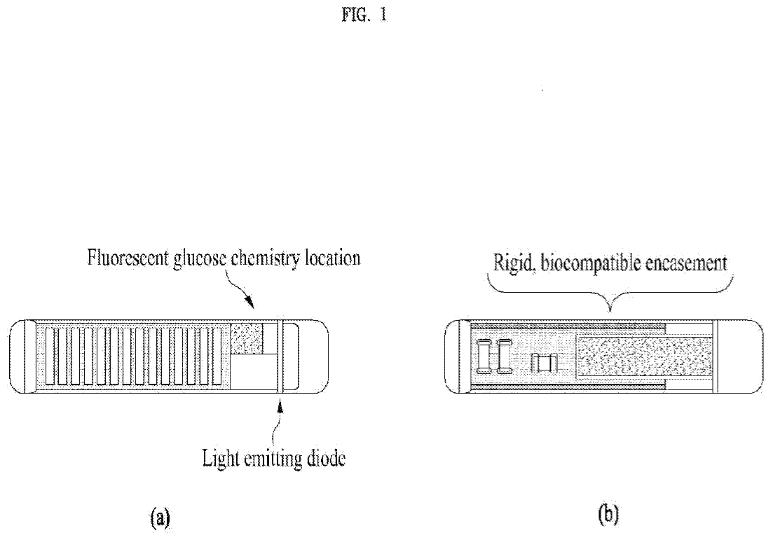

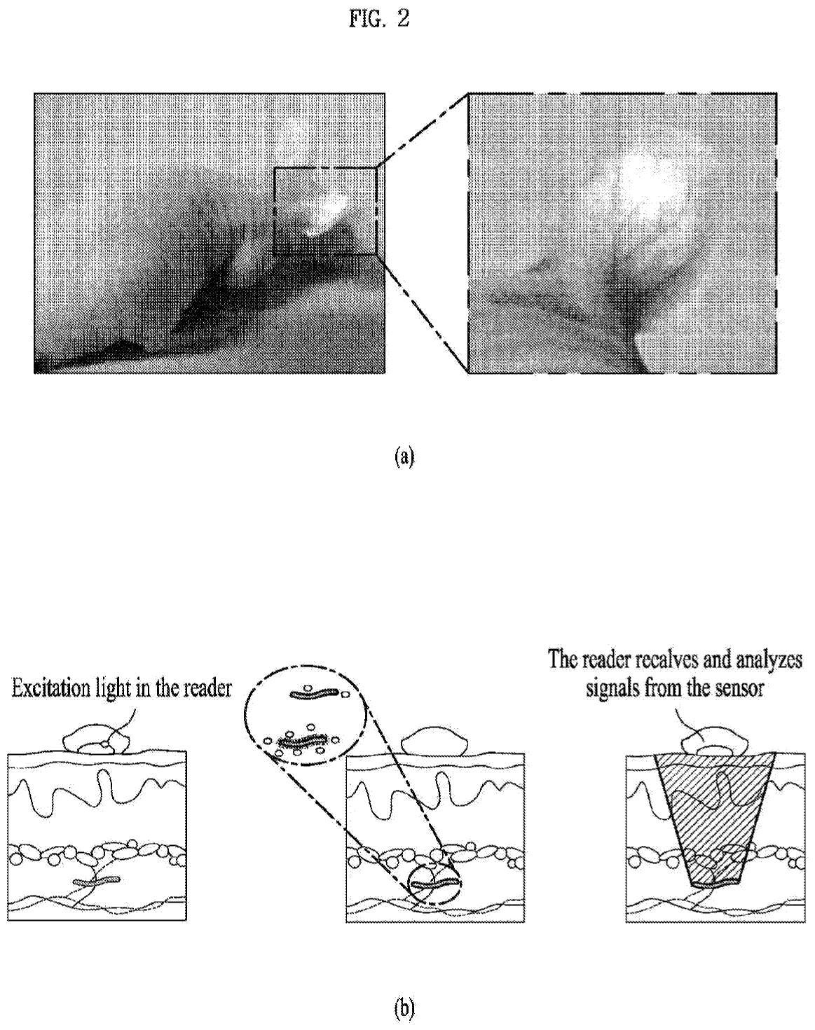

### BEST MODE

Hereinafter, an embodiment disclosed in the present specification will be described in detail with reference to the accompanying drawings. Regardless of the drawings, the same or similar components are assigned the same reference numerals, and duplicated descriptions thereof will be omitted. The suffixes “module” and “unit” for the components used in the following description are given or mixed in consideration of only the ease of writing of the specification, and do not have distinct meanings or roles by themselves. In addition, in describing the embodiment disclosed herein, when it is determined that a detailed description of a related known technology may obscure the gist of the embodiment disclosed herein, the detailed description thereof will be omitted. In addition, the accompanying drawings are only for easy understanding of the embodiment disclosed herein, and it should be understood that the technical idea disclosed herein is not limited by the accompanying drawings, and includes all changes, equivalents or substitutes included in the spirit and scope of the present disclosure.

Terms including ordinal numbers, such as first, second, and the like, may be used to describe various elements, but the components may not be limited by the above terms. The above terms are used only for the purpose of distinguishing one component from another.

In addition, it will be understood that when a component is referred to as being ‘connected to’ or ‘coupled to’ another component herein, it may be directly connected to or coupled to the other component, or one or more intervening components may be present. On the other hand, it will be understood that when a component is referred to as being ‘directly connected to’ or ‘directly coupled to’ another component herein, there are no other intervening components.

As used herein, the singular forms ‘a’ and ‘an’ are intended to include the plural forms as well, unless the context clearly indicates otherwise.

It should be understood that the terms ‘comprises’, ‘comprising’, ‘includes’, and ‘including’ when used herein, specify the presence of the features, numbers, steps, operations, components, parts, or combinations thereof described herein, but do not preclude the presence or addition of one or more other features, numbers, steps, operations, components, or combinations thereof.

A biosensor may be defined as a biological substance that may sense presence of any substance or energy. However, recently, the biosensor has been used in a broader sense, so that it is often difficult to distinguish the biosensor from an actual chemical sensor. Accordingly, the present disclosure may not be limited to the biosensor and may be broadly referred to as the chemical sensor.

The chemical sensor may be defined as a device that may inform the presence of the energy or the substance using one of human senses. Fluorescence, color change, and an electrochemical analysis method may be used as a method for sensing the substance or the energy to be analyzed.

An optical measurement scheme is a scheme of identifying presence of a specific substance by measuring an amount of light generated when a fluorescence sensor coupled to the specific substance is stimulated by light irradiated from a light source.

The optical measurement scheme has advantages such as excellent sensitivity to observe a signal even at 10⁻⁹ M concentration, a relatively simple measurement method, and the like.

However, the existing optical measurement scheme has a disadvantage of inserting and removing the fluorescence sensor into and from a body through an incision procedure, and has a problem of short period of use resulted from a limitation of a battery.

The present disclosure may omit the incision procedure by inserting the fluorescence sensor into the body using a microneedle, and solve the limitation in the period of use by exposing a portion where the battery is inserted outside the body.

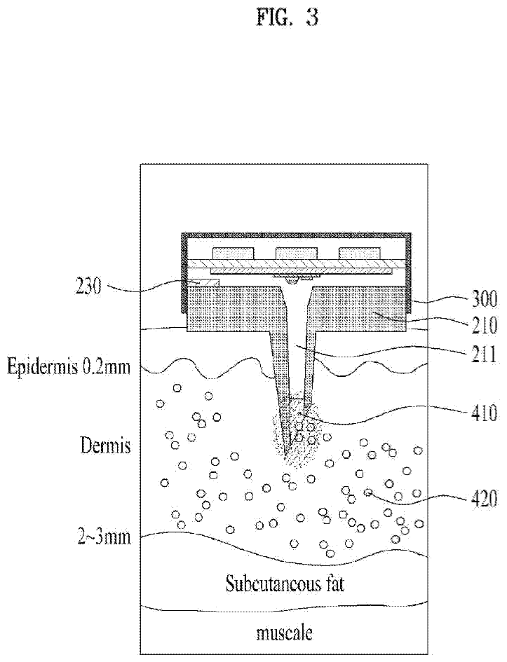

FIG. 3 shows an embodiment of a needle-type biosensor according to the present disclosure.

A needle-type biosensor of the present disclosure may include a hollow microneedle 210 including an internal flow channel 211 defined therein that is connected to a tip, a fluorescence sensor 410 that is disposed in the internal flow channel 211 and is coupled to a specific target substance 420 to fluoresce, and a reader 300 that senses the fluorescing of the fluorescence sensor.

The needle-type biosensor of the present disclosure may be attachable to skin. When the needle-type biosensor is attached to the skin, the microneedle 210 passes through epidermis and penetrates dermis, so that the fluorescence sensor 410 may react with the specific substance 420 present in the dermis.

The fluorescence sensor 410 of the present disclosure may be a substance that is coupled to and fluoresces with the specific substance 420. The most common scheme in which the fluorescence sensor 410 fluoresces is a scheme in which the fluorescence sensor 410 fluoresces as an ion or a substance to be analyzed is coupled thereto. However, in some cases, a scheme in which, when the fluorescence sensor 410 coupled to an indicator is coupled to the specific substance 420, the fluorescence sensor 410 fluoresces as the indicator is removed therefrom may be used. In some cases, instead of the fluorescence reaction, a light absorption reaction or a color change reaction scheme may be used.

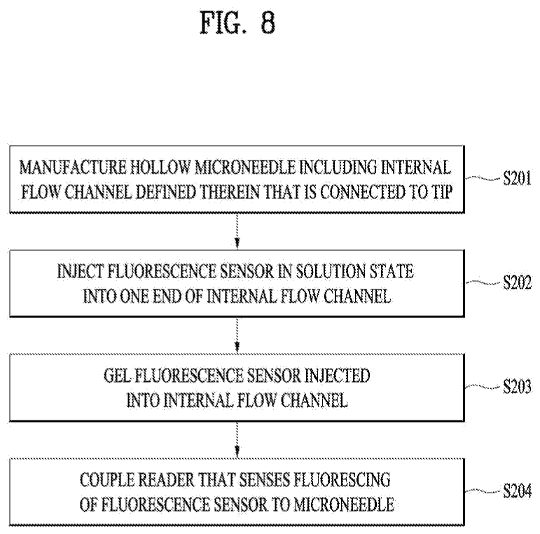

The fluorescence sensor 410 of the present disclosure may be introduced into the internal flow channel 211 in a solution state using a capillary phenomenon, and may be gelled and fixed. A scheme for fixing the fluorescence sensor 410 will be described in detail below with reference to FIG. 8.

The reader 300 of the present disclosure may sense presence and concentration of the specific substance 420 by sensing the light emitted from the fluorescence sensor 410.

The reader 300 of the present disclosure may be coupled to the microneedle 210 and may be exposed to the outside without being inserted into the skin. In some cases, the reader 300 may be detachably coupled to the microneedle 210, so that it is possible to replace the microneedle 210 as necessary. The microneedle 210 coupled to the reader 300 may include the different fluorescence sensor 410 depending on the specific substance 420 to be targeted. The microneedle 210 may include a monitoring portion 230 displaying information of the specific substance 420 to be targeted, and the reader 300 may acquire the information related to the specific substance 420 through the monitoring portion 230. Hereinafter, a detailed structure of the reader 300 will be described.

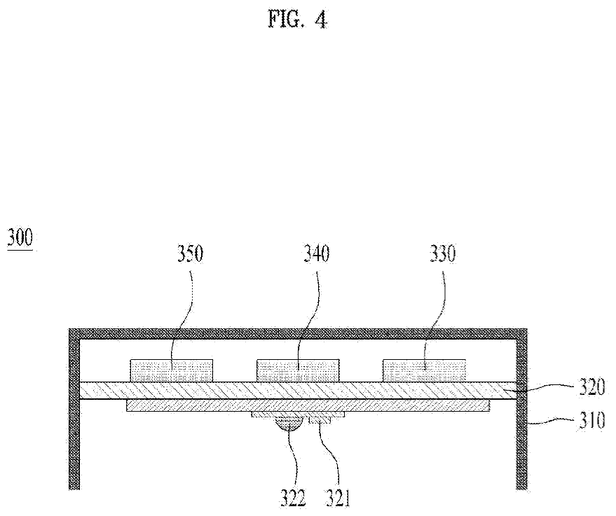

FIG. 4 is an enlarged view of the reader 300 in FIG. 3.

The reader 300 of the present disclosure may include a housing 310 that shields external light in the state in which the microneedle 210 is inserted into the skin, a light source 322 that is mounted in the housing 310 and irradiates light to the fluorescence sensor 410, and a light receiving portion 321 that receives light emitted from the fluorescence sensor 410 that fluoresces.

The light source 322 of the present disclosure may be a light-emitting diode (LED) that generates light having a specific wavelength.

The light receiving portion 321 of the present disclosure may include a photo diode, and may convert the light emitted as the fluorescence sensor 410 fluoresces into an electrical signal. The light emitted from the fluorescence reaction has a wavelength different from that of the light irradiated from the light source 322. Accordingly, the light receiving portion 321 may include a filter (not shown) that filters the light emitted from the fluorescence reaction.

The reader 300 of the present disclosure may include a printed circuit board 320 having the light source 322 and the light receiving portion 321, and the printed circuit board 320 may further include at least one of a driving IC 330, a micro controller unit (MCU) 340, a communication device 350, and a power supply (not shown).

The micro controller unit 340 of the present disclosure may drive the light source 322 and the light receiving portion 321 through the driving IC 330 in response to an external input. The external input may correspond to a power on/off device (not shown), and the on/off device may be disposed on an outer surface of the housing 310.

The micro controller unit 340 of the present disclosure may generate data corresponding to the presence or a standard quantity of the specific target substance 320 contained in the body through the electrical signal generated from the light receiving portion 321, and may transmit and receive the data through the communication device 350 that communicates with an external device in a wired or wireless manner.

The communication device 350 of the present disclosure may include one or more modules that enable wireless communication between the external device and the needle-type biosensor, between the needle-type biosensor and another needle-type biosensor, or between the needle-type biosensor and an external server. In addition, the communication device 350 may include one or more modules that connect the needle-type biosensor to one or more networks.

In this connection, the external device may be a general-purpose mobile device including a display unit for outputting data received through the communication device 350 and a memory for continuously storing the received data. In addition, the external device may be connected to an external server that provides a customized medical service through accumulated data to provide the customized medical service to a user with the needle-type biosensor according to the present disclosure attached.

The reader 300 of the present disclosure may further include at least one of a display that outputs the data, an alarm unit that outputs a warning sound, and a temperature sensor that senses a temperature on the outer surface of the housing 310.

The reader 300 of the present disclosure may receive the information displayed in the monitoring portion 230 disposed in the microneedle 210 through short-range wireless communication. In this connection, the information displayed in the monitoring portion 230 may include parameters corresponding to a type of the specific substance 420 to be targeted, an amount of light received based on the fluorescence response of the fluorescence sensor 410, and the standard quantity of the specific substance 420.

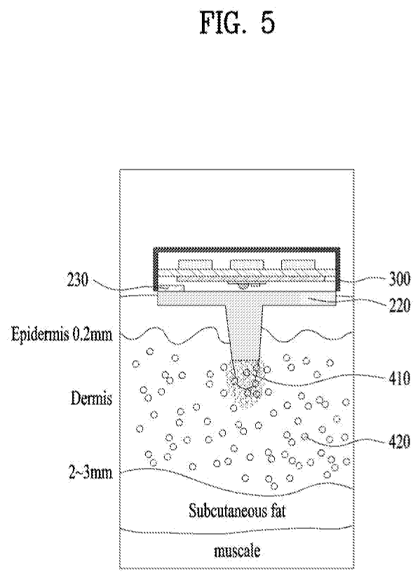

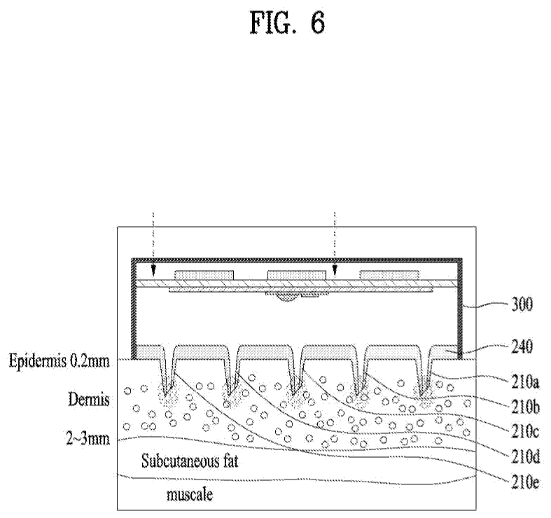

FIGS. 5 and 6 show other embodiments of a needle-type biosensor according to the present disclosure.

Specifically, FIG. 5 shows a needle-type biosensor including a solid microneedle 220.

The solid microneedle 220 of the present disclosure may be a microneedle that does not include the internal flow channel 211 unlike the microneedle 210 in FIG. 3, and has the fluorescence sensor 410 constituting the tip.

The solid microneedle 220 of the present disclosure may be made of a transparent material such that the light receiving portion of the reader 300 may sense the fluorescence reaction of the fluorescence sensor 410.

The solid microneedle 220 of the present disclosure may be fixed to the reader 300, and may be detachably attached to the reader 300 in some cases. The solid microneedle 220 may include the monitoring portion 230 displaying the information on the specific substance 420 reacting with the fluorescence sensor 410. The reader 300 of the present disclosure may receive the information displayed in the monitoring portion 230 through the short-range wireless communication. In this connection, the information displayed in the monitoring portion 230 may include the parameters corresponding to the type of the specific substance 420 to be targeted, the amount of light received based on the fluorescence response of the fluorescence sensor 410, and the standard quantity of the specific substance 420.

As another embodiment, FIG. 6 shows a needle-type biosensor including a multi-microneedle 240.

The multi-microneedle 240 of the present disclosure may be a microneedle including a plurality of microneedles 210a to 210e (210 in FIG. 3). In some cases, the multi-microneedle 240 may be a microneedle including a plurality of solid microneedles 220 in FIG. 5.

The multi-microneedle 240 of the present disclosure may include different fluorescence sensors respectively in the plurality of microneedles 210a to 210e. The different fluorescence sensors respectively in the plurality of microneedles 210a to 210 may be respectively coupled to different specific substances to fluoresce. The reader 300 may distinguish the type and the concentration of the specific substance in the body by distinguishing a microneedle that fluoresces among the plurality of microneedles 210a to 210e.

Because the needle-type biosensor in the present disclosure may be manufactured in a form of a pad that is attached to the skin, there is an advantage that a plurality of tests are possible through one pad when using the multi-microneedle 240.

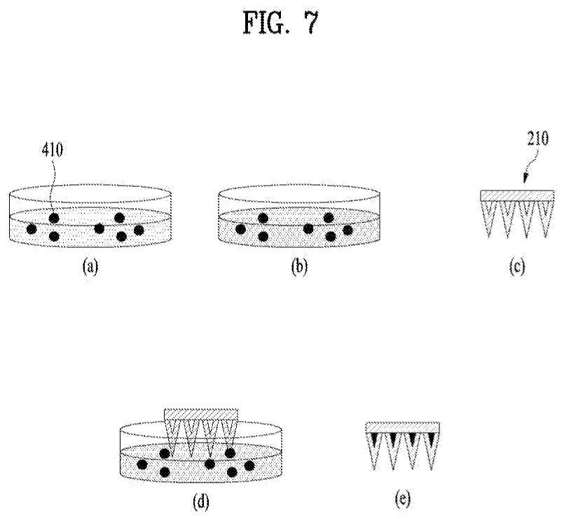

FIGS. 7 and 8 are views for illustrating a method for introducing a fluorescence sensor into a hollow microneedle in a needle-type biosensor according to the present disclosure. Hereinafter, a method for fixing the fluorescence sensor 410 to the hollow microneedle 210 will be described.

The microneedle 210 of the present disclosure includes an internal flow channel defined therein that is connected to a tip, and the internal flow channel includes a fluorescence sensor. The needle-type biosensor of the present disclosure may be attached to the skin such that the tip of the microneedle 210 is inserted into the body. The fluorescence sensor inserted into the body through the microneedle 210 may be coupled to a specific substance to fluoresce. In this connection, the fluorescence sensor needs to be fixed so as not to be removed from the microneedle 210 and introduced into the body.

The method for fixing the fluorescence sensor 410 of the present disclosure to the hollow microneedle 210 is as follows.

The fluorescence sensor 410 of the present disclosure is dissolved in a solvent to be in a solution state ((a) in FIG. 7). The fluorescence sensor 410 in the solution state is mixed with a gelable solution ((b) in FIG. 7). The internal flow channel of the microneedle 210 is subjected to a hydrophilic treatment such that the boronic-based fluorescence sensor may be easily introduced into the internal flow channel of the microneedle 210 ((c) in FIG. 7). The fluorescence sensor 410 is introduced into the internal flow channel that has been subjected to the hydrophilic treatment. In this connection, the fluorescence sensor 410 may be introduced into the internal flow channel of the microneedle 210 using a capillary phenomenon. In some cases, the fluorescence sensor 410 may be introduced into the internal flow channel by increasing an external pressure ((d) in FIG. 7). The fluorescence sensor 410 introduced into the internal flow channel of the microneedle 210 in the solution state may be gelled and fixed to the microneedle 210. The fluorescence sensor 410 introduced into the internal flow channel of the microneedle 210 in the solution state may be gelled using at least one of a temperature, a time, and a UV ((e) in FIG. 7).

A full process to manufacture the needle-type biosensor of the present disclosure is as follows.

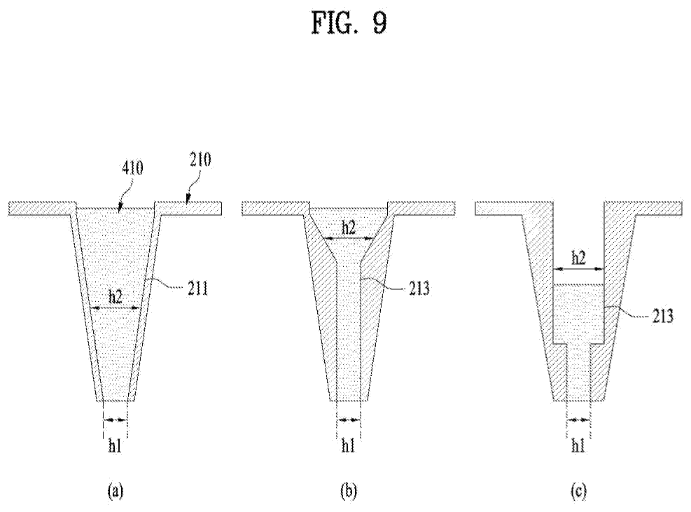

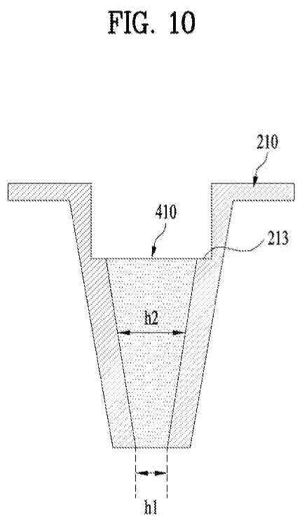

The present disclosure manufactures the hollow microneedle including the internal flow channel defined therein that is connected to the tip (S201). The microneedle of the present disclosure may be inserted into the body while the fluorescence sensor is introduced into the internal flow channel. The fluorescence sensor inserted into the body through the microneedle may be coupled to the specific substance in the body to fluoresce. In the microneedle of the present disclosure, the internal flow channel may be subjected to the hydrophilic treatment such that the fluorescence sensor in the liquid state may be easily introduced thereinto. The internal flow channel of the microneedle may have a structure in which the introduced fluorescence sensor is gelled and fixed. A description related thereto is made in detail with reference to FIGS. 9 and 10.

In the present disclosure, the fluorescence sensor may be dissolved in the solvent to be in the solution state, and the fluorescence sensor in the solution state may be mixed with the gelable solution. Thereafter, the fluorescence sensor in the solution state may be injected into the internal flow channel of the microneedle of the present disclosure (S202). The fluorescence sensor in the solution state may be introduced into the internal flow channel of the microneedle by the capillary action, or may be introduced into the internal flow channel using a difference in pressure. In this connection, because the internal flow channel of the microneedle is subjected to the hydrophilic treatment, the fluorescence sensor having the same property may be easily introduced into the internal flow channel.

The fluorescence sensor of the present disclosure may be gelled and fixed in the state of being introduced into the internal flow channel of the microneedle (S203). The fluorescence sensor in the liquid state may be gelled by changing the temperature, passing the time, or irradiating light of a specific wavelength. The gelled fluorescence sensor may be fixed to the internal flow channel of the microneedle by a frictional force. In addition, the microneedle of the present disclosure may have the structure to which the fluorescence sensor may be fixed. This will be described in detail with reference to FIGS. 9 and 10.

The microneedle of the present disclosure may be coupled to the reader that induces and senses the fluorescence response of the fluorescence sensor (S204). The needle-type biosensor of the present disclosure may be attached to the skin in the form of the patch with the microneedle inserted into the body and the reader exposed to the outside. Because the needle-type biosensor of the present disclosure is used by being attached to the skin in the form of the patch, there is no need for the separate incision procedure. In addition, in the present disclosure, when the microneedle to be inserted into the body is attached with a microscopic size, pain may be reduced. In addition, the needle-type biosensor of the present disclosure includes a battery replacement portion in the reader, so that the battery may be replaced even when the needle-type biosensor is attached to the skin.

Hereinafter, the structure of the internal flow channel for fixing the gelled fluorescence sensor to the microneedle of the present disclosure will be described.

FIG. 9 is a view for illustrating a structure of a hollow microneedle for fixing an introduced fluorescence sensor in a needle-type biosensor according to the present disclosure.

The present disclosure relates to the needle-type biosensor. The needle-type biosensor may include the hollow microneedle 210 including the internal flow channel 211 defined therein that is connected to the tip, the fluorescence sensor 410 that is disposed in the internal flow channel 211 and is coupled to the specific target substance to fluoresce, and the reader 300 (see FIG. 3) that senses the fluorescing of the fluorescence sensor 410.

The internal flow channel 410 of the present disclosure may include a section with a diameter greater than a diameter h1 of one end connected to the tip of the microneedle 210.

Specifically, (a) in FIG. 9 shows an embodiment in which a diameter of the internal flow channel 211 of the present disclosure gradually increases from the tip. The internal flow channel 410 of the present disclosure may have an internal diameter h2 greater than the diameter h1 of the tip to have an inverted triangle structure. The fluorescence sensor 410 disposed in the internal flow channel 410 may be gelled along the inverted triangular shape of the internal flow channel 211 and fixed to the internal flow channel 211. That is, in the present disclosure, the internal flow channel 211 has the inverted triangular shape with respect to the tip to prevent the fluorescence sensor 410 from leaking into the body.

As another embodiment, (b) in FIG. 9 shows that the internal flow channel 211 has a section 213 having a constant diameter from the tip, and then, has the diameter h2 that is greater than the diameter h1 of the tip. When the internal flow channel 211 has the section 213 having the constant diameter from the tip, the fluorescence sensor 410 in the liquid state may be more easily introduced into the internal flow channel 211 by the capillary action.

As another embodiment, in (c) in FIG. 9, the diameter of the internal flow channel 211 may vary discontinuously. That is, the internal flow channel 211 of the present disclosure may include a step at which the internal diameter varies discontinuously. Considering that the microneedle 210 has the microscopic size, an increase in the diameter of the internal flow channel using the step may facilitate the manufacturing process.

FIG. 10 is a view for illustrating a structure of a hollow microneedle for quantitatively introducing a fluorescence sensor in a needle-type biosensor according to the present disclosure.

In the present disclosure, the reader 300 (FIG. 3) may sense the fluorescence reaction of the fluorescence sensor 410 to sense the presence of the internal specific substance, and sense the concentration of the specific substance through a degree of the fluorescence reaction. In order to sense the concentration of the specific substance through the degree of the fluorescence reaction of the fluorescence sensor 410 by the reader 300, the fluorescence sensor 410 needs to be quantitatively introduced into the internal flow channel 211 and fixed.

The present disclosure may include a step 213 at which the internal diameter varies discontinuously such that the fluorescence sensor 410 may be quantitatively introduced into the internal flow channel 211. The fluorescence sensor 410 in the liquid state may be introduced from the tip into the internal flow channel 211 through the capillary action. In this connection, the fluorescence sensor 410 introduced into the internal flow channel 211 is continuously introduced up to the step 213 at which the diameter varies discontinuously, but the inflow phenomenon may be stopped near the step 213. In the present disclosure, the fluorescence sensor 410 may be quantitatively fixed to the microneedle by disposing the step 213 at a preset distance from the tip. The present disclosure may sense the concentration of the specific substance through the degree of fluorescence reaction by fixing the fluorescence sensor 410 quantitatively to the microneedle 210.

The present disclosure may include the step 213 on the internal flow channel 211 of the microneedle 210 to quantitatively introduce the fluorescence sensor 410, and may include a section in which the diameter increases between the tip and the step 213 to fix the gelled fluorescence sensor 410 to the internal flow channel 211. Specifically, FIG. 10 shows an embodiment in which the diameter h1 of the tip continuously increases in the internal flow channel 211. However, the internal diameter of the internal flow channel 211 may increase from the tip to the step 213 as shown in FIG. 9.

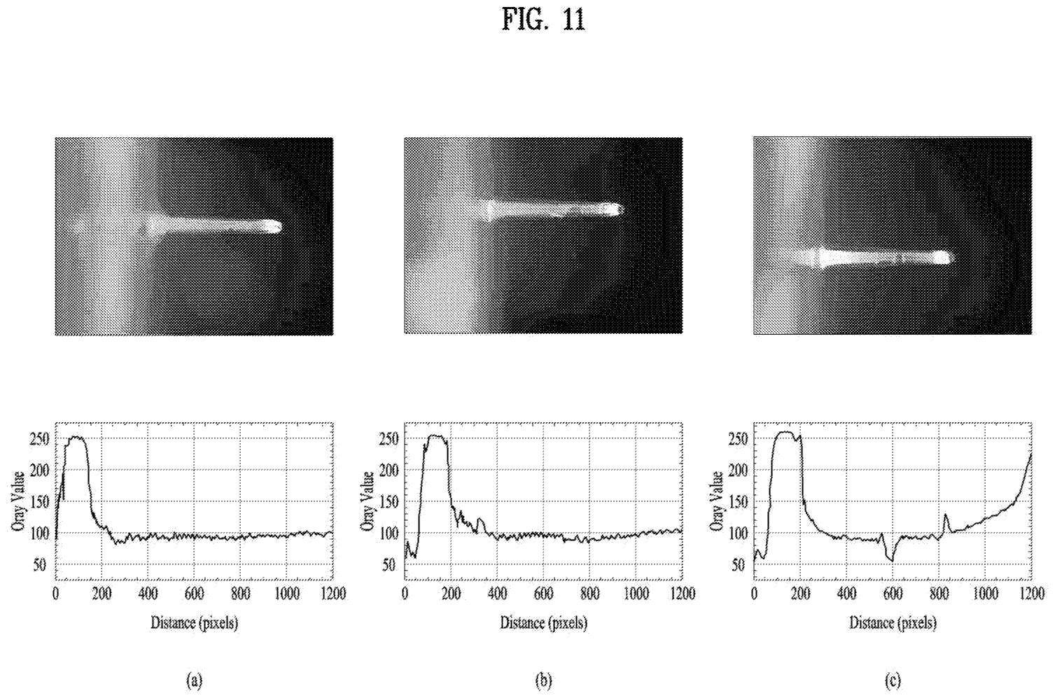

FIG. 11 is a view for illustrating data sensed through a reader based on a degree of light emission of a fluorescent substance in a needle-type biosensor according to the present disclosure.

Because the microneedle 211 of the present disclosure has the fluorescence sensor 410 quantitatively, data of an experiment of measuring the concentration of the specific substance by sensing the degree of the fluorescence reaction is shown.

Specifically, (a) to (c) in FIG. 11 show a degree of luminescence of the microneedle based on the concentration of the specific substance in the body and the data acquired from the reader. The present disclosure may identify the presence and the concentration of the specific substances in the body through the data acquired through the reader.

The reader of the present disclosure may measure the luminescence degree of the fluorescence sensor in real time to measure the concentration of the specific substance in the body in real time and provide the measured data to the user in real time.

The specific target substance, which is a continuous measurement target, may be at least one of glucose, cholesterol, an inflammatory response marker, and a pecking immune response marker.

The above detailed description should not be construed as restrictive in all respects and should be considered as illustrative. The scope of the present disclosure should be determined by a reasonable interpretation of the appended claims, and any changes within the equivalent scope of the present disclosure are included in the scope of the present disclosure.

1. A needle-type biosensor comprising:
a hollow microneedle having an internal flow channel defined therein connected to a tip thereof;
a fluorescence sensor disposed in the internal flow channel, wherein the fluorescence sensor is coupled to a specific target substance to fluoresce; and
a reader for sensing the fluorescing of the fluorescence sensor,
wherein the internal flow channel includes a section with a diameter increasing to be greater than a diameter of one end of the internal flow channel connected to the tip.

2. The needle-type biosensor of claim 1, wherein the fluorescence sensor is gelled along a shape of the internal flow channel after being injected into the internal flow channel from said one end of the internal flow channel in a solution state, wherein the fluorescence sensor is fixed to the internal flow channel by a difference in diameter of the internal flow channel.

3. The needle-type biosensor of claim 1, wherein the internal flow channel includes a step where an internal diameter thereof varies discontinuously.

4. The needle-type biosensor of claim 3, wherein the internal flow channel includes the step at a location a preset distance away from said one end of the internal flow channel,
wherein the fluorescence sensor is disposed from said one end of the internal flow channel to the step.

5. The needle-type biosensor of claim 4, wherein the internal flow channel has the section with the increasing diameter between said one end of the internal flow channel and the step.

6. A method for manufacturing a needle-type biosensor, the method comprising:
manufacturing a hollow microneedle including an internal flow channel defined therein connected to a tip thereof;
injecting a fluorescence sensor in a solution state into one end of the internal flow channel; and
gelling and fixing the fluorescence sensor injected into the internal flow channel.

7. The method of claim 6, wherein the manufacturing of the hollow microneedle includes performing a hydrophilic treatment on the internal flow channel.

8. The method of claim 6, wherein the injecting of the fluorescence sensor includes injecting the fluorescence sensor in the solution state into the internal flow channel through a capillary phenomenon.

9. The method of claim 6, wherein the manufacturing of the hollow microneedle includes forming a section with a diameter increasing to be greater than a diameter of said one end of the internal flow channel connected to the tip.

10. The method of claim 8, wherein the gelling and fixing of the fluorescence sensor includes gelling the fluorescence sensor in the solution state using at least one of a temperature, a time, and a UV,
wherein the internal flow channel prevents the gelled fluorescence sensor from leaking with a difference in diameter of the internal flow channel.

11. The method of claim 8, wherein the manufacturing of the hollow microneedle includes forming a step where an internal diameter of the internal flow channel varies discontinuously,
wherein the injecting of the fluorescence sensor includes injecting the fluorescence sensor from said one end of the internal flow channel to the step.
